(19) United States
(12) Patent Application Publication
(10) Pub. No.: US 2023/0384810 A1
(43) Pub. Date: Nov. 30, 2023

(54) DEVICE AND ENERGIZING METHOD

(71) Applicant: TRIPOD DESIGN CO., LTD., Tokyo (JP)

(72) Inventor: Satoshi NAKAGAWA, Tokyo (JP)

(73) Assignee: TRIPOD DESIGN CO., LTD., Tokyo (JP)

(21) Appl. No.: 18/032,421

(22) PCT Filed: Oct. 19, 2021
(86) PCT No.: PCT/JP2021/038663

§ 371 Date: Apr. 18, 2023

(30) Foreign Application Priority Data
Oct. 21, 2020 (JP) 2020-176739
Oct. 21, 2020 (WO) PCT/JP2021/038663

(51) Int. Cl. G05F 1/56; A61N 1/378; G01R 19/165; G01R 27/02

(52) U.S. Cl. CPC G05F 1/56; A61N 1/378; G01R 19/16533; G01R 27/02

(57) ABSTRACT

A device capable of operating on a standalone power source includes: a first conductive part and a second conductive part; and a functional part, wherein the first conductive part and the functional part are connected to each other, the second conductive part and the functional part are connected to each other, the first conductive part and the second conductive part are not in contact with each other; and the device is energized by bringing the first conductive part and the second conductive part into contact with a body.

### TECHNICAL FIELD

The present invention relates to a device operating on a standalone power source.

### BACKGROUND ART

In recent years, devices equipped with a function capable of measuring a heart rate or the like, such as a smart watch, have been widely used.

However, a device equipped with a function capable of measuring a heart rate or the like has a limited time during which the device can be continuously operated, and it has been necessary to perform charging depending on a use situation.

### SUMMARY OF INVENTION

### Technical Problem

At least one object of the present invention is to provide a device capable of operating on a standalone power source.

### Solution to Problem

The present invention solves the above problem by any of the following [1] to [10].

[1] A device comprising: a first conductive part and a second conductive part; and a functional part, wherein the first conductive part and the functional part are connected to each other, the second conductive part and the functional part are connected to each other, the first conductive part and the second conductive part are not in contact with each other; and the device is energized by bringing the first conductive part and the second conductive part into contact with a body;

[2] The device according to [1], comprising a voltage boost circuit, wherein an electromotive force generated between the first conductive part and the second conductive part is boosted by the voltage boost circuit;

[3] The device according to [1] or [2], wherein the first conductive part and the second conductive part have flexibility;

[4] The device according to any one of [1] to [3], comprising a measurement part that measures an internal impedance of the device and/or a predetermined voltage in the device;

[5] The device according to any one of [1] to [3], comprising a predetermined sensor, wherein the sensor is operated by bringing the first conductive part and the second conductive part into contact with the body to be energized;

[6] The device according to [4] or [5], comprising a communication part that transmits to another computer device the internal impedance of the device and/or the predetermined voltage in the device measured by the measurement part, or information acquired by the predetermined sensor;

[7] The device according to any one of [1] to [6], wherein the first conductive part has a standard electrode potential different from that of the second conductive part;

[8] The device according to any one of [1] to [7], comprising a fixing part for fixing the first conductive part and the second conductive part in a state of being in contact with the body;

[9] The device according to any one of [1] to [8], comprising an electrical stimulation generator that generates a current for giving an electrical stimulation to the body by a voltage generated by bringing the first conductive part and the second conductive part into contact with the body;

[10] An energizing method comprising energizing a device, the device including a first conductive part and a second conductive part, and a functional part, the first conductive part and the functional part being connected to each other, the second conductive part and the functional part being connected to each other, the first conductive part and the second conductive part being not in contact with each other, by bringing the first conductive part and the second conductive part into contact with a body.

### Advantageous Effects of Invention

According to the present invention, it is possible to provide a device capable of operating on a standalone power source.

### BRIEF DESCRIPTION OF DRAWINGS

FIG. 1 is a block diagram illustrating a configuration of a device according to an embodiment of the present invention.

FIG. 2 is a block diagram illustrating a configuration of an electric power conversion part according to an embodiment of the present invention.

FIG. 3 is a diagram illustrating a device according to an embodiment of the present invention.

FIG. 4 is a diagram illustrating a relationship between time and a current I in a case where ON/OFF of a transistor in the device is switched according to an embodiment of the present invention.

FIG. 5 is a diagram illustrating an example of a device according to an embodiment of the present invention.

FIG. 6 is a diagram illustrating an example of a device according to an embodiment of the present invention.

### DESCRIPTION OF EMBODIMENTS

Hereinafter, embodiments of the present invention will be described with reference to the accompanying drawings. Hereinafter, the description regarding effects is one aspect of the effects of the embodiments of the present invention, and is not limited to the description herein.

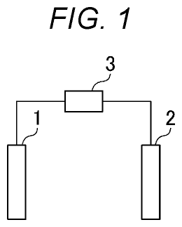

FIG. 1 is a block diagram illustrating a configuration of a device according to an embodiment of the present invention. As illustrated in FIG. 1, the device includes a first conductive part 1, a second conductive part 2, and a functional part 3. The first conductive part 1 and the functional part 3 are electrically connected to each other, and the functional part 3 and the second conductive part 2 are electrically connected to each other. The term “electrically connected” means, for example, energizably connected with a conductive wire or the like.

The first conductive part 1 and the second conductive part 2 of the device are not in contact with each other. The term “not in contact” refers to, for example, a state in which the first conductive part 1 and the second conductive part 2 are not in direct contact with each other.

The device energizes the first conductive part 1 and the second conductive part 2 by bringing the first conductive part 1 and the second conductive part 2 into contact with a body. This is because by bringing the first conductive part 1 and the second conductive part 2 into contact with the body, a part or all of the first conductive part 1 and the second conductive part 2 come into contact with a medium to be described later.

The distance between the first conductive part 1 and the second conductive part 2 is preferably 5 mm or less, more preferably 3 mm or less, still more preferably 1 mm or less, notably preferably 0.5 mm or less, particularly preferably 0.3 mm or less, specially preferably 0.1 mm or less, and most preferably 0.05 mm or less. The distance between the first conductive part 1 and the second conductive part 2 may be constant or partially different. When the distance between the first conductive part 1 and the second conductive part 2 is partially different, the distance of the closest part among the distances between the first conductive part 1 and the second conductive part 2 is preferably within the above range. Furthermore, when the distance between the first conductive part 1 and the second conductive part 2 is partially different, the average value of the distances between the first conductive part 1 and the second conductive part 2 is preferably within the above range. When the distance between the first conductive part 1 and the second conductive part 2 is within the above range, the first conductive part 1 and the second conductive part 2 can be efficiently in contact with the medium, and the device is easily energized.

It is preferable that both the first conductive part 1 and the second conductive part 2 have conductivity. Here, examples of the material of the first conductive part 1 and the second conductive part 2 include metal, conductive polymers, carbon, conductive fibers, conductive rubbers, and the like.

Shapes of the first conductive part 1 and the second conductive part 2 are not particularly limited. The first conductive part 1 and the second conductive part 2 may have a rectangular parallelepiped shape, a round columnar shape (rod shape), a pyramid shape, a conical shape, a plate shape, a sheet shape, a film shape, a string shape, or powder form, and may have any shape.

Furthermore, for the first conductive part 1 and the second conductive part 2, a nonconductive material covered with a conductive material, a nonconductive material mixed with a conductive material, or the like may be used. For example, a plastic film coated with a metal or cream-like paste mixed with metal powder may be used. The first conductive part 1 and the second conductive part 2 may have flexibility.

The metal used for the first conductive part 1 and the second conductive part 2 can be appropriately selected from, for example, silver, copper, gold, aluminum, magnesium, zinc, nickel, platinum, tin, titanium, stainless steel, zinc oxide, magnesium oxide, oxides of the above-described metals, and the like. In addition, a predetermined metal may be coated with another metal different from the predetermined metal or another material having conductivity.

Materials of the first conductive part 1 and the second conductive part 2 may be of different type from each other, or may be of the same type as each other. For example, sheet-shaped stainless steel can be used for the first conductive part 1, and sheet-shaped zinc can be used for the second conductive part 2. In this case, the first conductive part 1 and the second conductive part 2 are connected to the functional part 3 or a voltage boost circuit/voltage step-down circuit with a conductive wire.

When polarization resistance is measured for at least one of the first conductive part 1 or the second conductive part 2 using an AC impedance method, the measured value is preferably 100Ω or more.

Here, a conductive part serving as a starting point of a current is defined as the first conductive part 1, and a conductive part serving as an ending point is defined as the second conductive part 2. Which conductive part functions as the first conductive part 1 is determined by a material of the conductive part or an environment surrounding the conductive part (for example, temperature, humidity, atmospheric pressure, pH, and the like). A chemical reaction occurs at an interface between the first conductive part 1 or the second conductive part 2 and the medium 4, and free electrons are generated in the conductive part.

For example, when different metals are used for the first conductive part 1 and the second conductive part 2, the conductive part made of a metal having a lower standard electrode potential is used as the first conductive part 1, and the conductive part made of a metal having a higher standard electrode potential is used as the second conductive part 2. In this case, electrons move from the second conductive part 2 toward the functional part 3, and electrons move from the functional part 3 toward the first conductive part 1. That is, a current is generated from the first conductive part 1 side to the second conductive part 2 side via the functional part 3. For example, in the second conductive part 2, the metal constituting the conductive part is eluted as a cation into the medium 4 to generate free electrons, and in the first conductive part 1, the cation in water of the medium 4 reacts with the electrons to be electrically neutralized.

The level of the standard electrode potential is determined by comparing relative values (relative values) of the standard electrode potentials of substances, and is not determined by using an absolute value of the standard electrode potential. For example, when substance A having a standard electrode potential of −5 V is compared with substance B having a standard electrode potential of +2 V, the standard electrode potential of substance A is low, and the standard electrode potential of substance B is high.

On the other hand, even when the same metal is used for the conductive parts, either one of the conductive parts functions as the first conductive part 1 and the other conductive part functions as the second conductive part 2 depending on the conditions of the surrounding environment of the conductive parts, such as temperature, humidity, atmospheric pressure, and pH, for example, and a current is generated. Therefore, when conditions such as ambient temperature, humidity, atmospheric pressure, and pH of the two conductive parts are changed, the one functioning as the first conductive part may function as the second conductive part, and the one functioning as the second conductive part may function as the first conductive part.

The electromotive force generated from the first conductive part 1 and the second conductive part 2 is preferably 0.9 V or less, more preferably 0.35 V or less, and still more preferably 0.25 V or less. The electromotive force generated from the first conductive part 1 and the second conductive part 2 is preferably 5 mV or more.

Furthermore, although not illustrated, the device may include a plurality of first conductive parts 1 and a plurality of second conductive parts 2. For example, a plurality of first conductive parts 1a, 1b to 1n (n is an integer of 2 or more) may be electrically connected in parallel. In addition, a plurality of second conductive parts 2a, 2b to 2m (m is an integer of 2 or more) may be electrically connected in parallel. Note that the plurality of first conductive parts 1a, 1b to 1n may be electrically connected in series. Furthermore, the plurality of second conductive parts 2a, 2b to 2m may be electrically connected in series.

The functional part 3 is, for example, a part that executes a predetermined function by energization. The functional part 3 can include an electric consumption part that consumes electric power and exerts a predetermined function, an electric storage part that stores electricity generated in a conductive part, an output voltage conversion part or the like that converts a voltage to be output, such as a voltage boost circuit and a voltage step-down circuit, a control part such as a microcomputer that controls a circuit, a communication part capable of wirelessly communicating with other devices, and a display part that displays information, and the like.

As the electric consumption part, for example, any of a light source such as an incandescent light bulb or a light emitting diode, a heat generator that emits heat, a sounding body that emits sound, a transmitter that emits a signal, a sensor that detects predetermined information, and the like can be adopted. The electric storage part may be included in the voltage boost circuit or the voltage step-down circuit. A control part such as a microcomputer can control a circuit to release the electricity stored in the electric storage part under a predetermined condition. The released electricity is consumed by the electric consumption part. In addition, even in the control part such as a microcomputer, electric power is consumed slightly, and thus, it is possible to perform control so as to release the stored electricity while securing electric power necessary for activating the control part.

The functional part 3 may include any one of the electric consumption part, the electric storage part, the output voltage conversion part, the communication part, the display part, and the control part, and the functional part 3 may be formed by combining any two or more of the power consumption part, the electric storage part, the output voltage conversion part, the communication part, the display part, and the control part. In addition, the functional part 3 may be formed by integrally forming any two or more of the electric consumption part, the electric storage part, the output voltage conversion part, the communication part, the display part, and the control part, or may be formed by separately forming any of the electric consumption part, the electric storage part, the output voltage conversion part, the communication part, the display part, and the control part while electrically connecting them.

Input impedance in the functional part 3 is preferably 1 kΩ or more, and more preferably 10 kΩ or more. In addition, the input impedance of the functional part 3 preferably has a non-linear current-voltage characteristic (I-V characteristic). The non-linear current-voltage characteristic refers to, for example, a case where, in a voltage change when a current flows through the functional part 3, the voltage value increases as a current value increases, but as the current value increases, an increase width of the voltage value required to increase the current value increases, and the voltage is not proportional to the current. In other words, the current value increases as the voltage value applied to the functional part 3 increases, but the degree of increase in the current value, which is increased by an increase in the voltage value, decreases as the voltage value increases, and the current value is not proportional to the voltage value. Since the input impedance in the functional part 3 has the non-linear current-voltage characteristic, the electromotive force generated between the first conductive part 1 and the second conductive part 2 is easily maintained.

The functional part 3 preferably has a function of converting output impedance. As a result, an influence on an input signal of the functional part 3 can be controlled.

In addition, the functional part 3 includes the electric storage part, and stores electric charge supplied from the first conductive part and/or the second conductive part. The control part performs control to release the stored electric charge in a time shorter than a time required to store the electric charge.

The lower limit value of the operating voltage of the functional part 3 is preferably 0.9 V or less. It is more preferable to operate at 0.35 V or less, and it is still more preferable to operate at 20 mV or less.

The medium is not particularly limited as long as it can cause a chemical reaction with the first conductive part 1 or the second conductive part 2 and ionizes. For example, the medium includes sweat and the like. The main component of sweat is water. The sweat may be liquid or gas after being evaporated. In addition, sweat may contain an electrolyte, lactate, urea, sebum, trace elements, and the like. Furthermore, sweat may include foreign substances such as mud, soil, and sand.

Among electrolytes contained in the medium, the concentration of cations may be 1 mol/L or less, 0.6 mol/L or less, 0.1 mol/L or less, 0.01 mol/L or less, or 0.001 mol/L or less.

Resistance value of the medium between the first conductive part 1 and the second conductive part 2 is preferably 1 kΩ or more, and more preferably 10 kΩ or more.

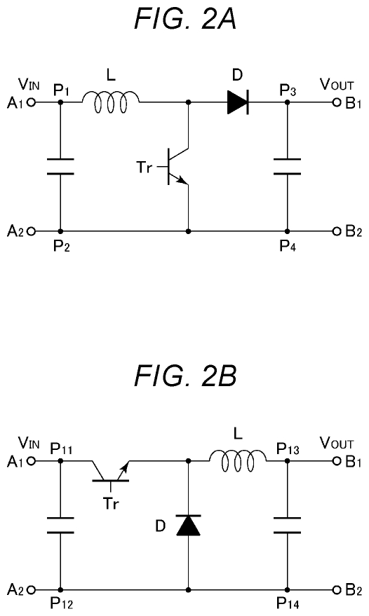

FIG. 2 is a block diagram illustrating a configuration of an electric power conversion part according to an embodiment of the present invention. FIG. 2A is a circuit diagram of a voltage boost circuit according to an embodiment of the present invention. A voltage boost circuit or a voltage step-down circuit is an example of the functional part 3, and includes an electric storage part.

As illustrated, an inductor L, a diode D, a transistor Tr, and a capacitor C are electrically connected. For example, an input terminal A1 is connected to the first conductive part 1, and an input terminal A2 is connected to the second conductive part 2. An output terminal B1 and an output terminal B2 are connected to an electric consumption part, a control part, and the like. The control part may be connected in parallel with the voltage boost circuit between the voltage boost circuit, and the first conductive part 1 and the second conductive part 2.

When an input voltage V_{IN} is applied while the transistor Tr is ON, electric energy is stored in the inductor L. The input voltage V_{IN} is the potential difference between a connection point P₁ and a connection point P₂. When the transistor Tr is OFF, the energy stored in the inductor L is added to the electric energy derived from the input voltage V_{IN}, and is output via the diode D. As a result, an output voltage V_{OUT}, which is the potential difference between a connection point P₃ and a connection point P₄, is higher than the input voltage V_{IN}. The voltage boost circuit may be based on the premise that the input voltage V_{IN} is a voltage lower than a predetermined voltage, and boost control may not be executed at a voltage higher than the predetermined voltage. The input voltage V_{IN} of the voltage boost circuit is preferably 5 mV or more. Note that ON/OFF of the transistor Tr is controlled by the control part.

FIG. 2B is a circuit diagram of a voltage step-down circuit according to an embodiment of the present invention. As illustrated, the transistor Tr, the inductor L, the diode D, and the capacitor C are electrically connected. For example, the input terminal A1 is connected to the first conductive part 1, and the input terminal A2 is connected to the second conductive part 2. The output terminal B1 and the output terminal B2 are connected to the electric consumption part, the control part, and the like. The control part may be connected in parallel with the voltage step-down circuit between the voltage step-down circuit, and the first conductive part 1 and the second conductive part 2.

When the transistor Tr is ON, electric energy is stored in the inductor L. The input voltage V_{IN} is the potential difference between a connection point P₁₁ and a connection point P₁₂, and the output voltage V_{OUT} is the potential difference between a connection point P₁₃ and a connection point P₁₄. In this case, the input voltage V_{IN} is substantially equal to the output voltage V_{OUT}. When the transistor Tr is OFF, the potential of at a connection point P₁₅ at the left end of the inductor L becomes lower than the potential at the connection point P₁₄, so that the output voltage V_{OUT} becomes a lower voltage. The voltage step-down circuit may be based on the premise that the input voltage V_{IN} is higher than a predetermined voltage, and step-down control may not be executed at a voltage lower than the predetermined voltage. Note that ON/OFF of the transistor Tr is controlled by the control part.

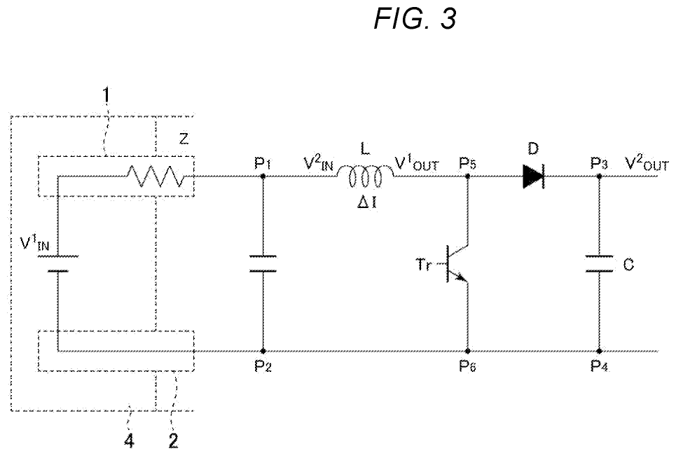

Next, a method for measuring the internal impedance of a system of the present invention will be described. FIG. 3 is a diagram illustrating a device according to an embodiment of the present invention. The potential difference between the first conductive part 1 and the second conductive part 2 can be defined as V¹_{IN}, and the potential difference between the connection point P₁ and the connection point P₂ can be defined as V²_{IN}. The potential difference between a connection point P₅ and a connection point P₆ can be defined as V¹_{OUT}, and the potential difference between the connection point P₃ and the connection point P₄ can be defined as V²_{OUT}. When the device of the present invention is energized, a current I flows between the first conductive part 1 and the second conductive part 2 in the direction of the connection point P₁ and the connection point P₅ by electromotive force V¹_{IN}.

As illustrated in FIG. 3, a voltage boost circuit is connected to the first conductive part 1 at the connection point P₁, and to the second conductive part 2 at the connection point P₂. In the voltage boost circuit, the inductor L, the diode D, the transistor Tr, and the capacitor C are electrically connected.

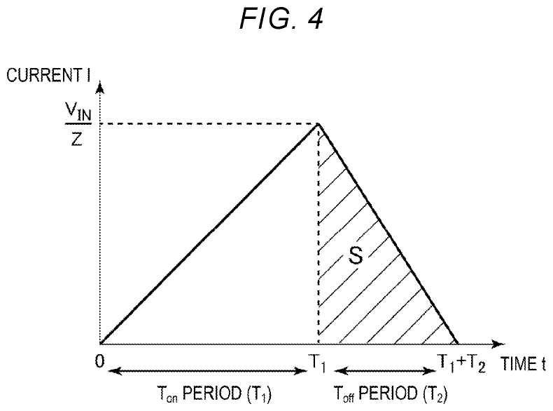

FIG. 4 is a diagram illustrating a relationship between time and the current I when ON/OFF of the transistor in the device is switched according to an embodiment of the present invention. Here, the relationship between V¹_{OUT} and V²_{IN} can be expressed by Equation (1): V¹_{OUT}−V²_{IN}=−L1×dI/dt using the current I flowing through the inductor L and inductance L1. When the transistor Tr is ON, since V¹_{OUT}=0, Equation (2): V²_{IN}=L1×dI/dt can be derived. In this case, dI/dt is a positive value, and the current I increases with time. On the other hand, when the transistor Tr is OFF, since V¹_{OUT}>V²_{IN} is satisfied, it can be seen from Equation (1): V¹_{OUT}−V²_{IN}=−L1×dI/dt that dI/dt is a negative value. In this case, the current I decreases with time. The ON and OFF of the transistor Tr are periodically repeated.

Here, when the first conductive part 1, the second conductive part 2, and the medium are regarded as one type of battery, it can be considered that the current I flows due to the electromotive force V¹_{IN}. In this case, when the internal impedance caused by the medium is defined as Z, the relationship between an input voltage and the internal impedance can be expressed by Equation (3): V¹_{IN}=Z×I+V²_{IN}.

In addition, while the transistor Tr is OFF (hereinafter, referred to as a T_{OFF} period), the capacitor C is charged with electric charge Q by the current I. Assuming that the voltage increased at the connection point P₃ during the T_{OFF} period is ΔV and the capacitor capacitance of the capacitor C is C1, Equation (4): Q=∫Idt=C1×ΔV holds.

From Equations (2) and (3), V¹_{IN}=L1×dI/dt+Z×I is derived. By solving this equation, Equation (5): I(t)=V¹_{IN}/Z+A×e^{(-Z/L1×t)} is derived, where A is an integral constant. In a case where the time when the transistor Tr is switched from OFF to ON is t=0, as is clear from FIG. 4, the current I is zero when t=0. Therefore, when t=0 and I=0 are substituted into Equation (5), it is found that the relationship of A=−V¹_{IN}/Z holds. When this A=−V¹_{IN}/Z is substituted into Equation (5), Equation (6): I(t)=V¹_{IN}/Z×(1−e^{(-Z/L1×t)} can be derived. The current I while the transistor Tr is ON (hereinafter, referred to as a Tₒₙ period) can be calculated by Equation (6). When a time during which the transistor Tr is ON is sufficiently taken, the maximum value of the current I is V¹_{IN}/Z.

The current I when the Tₒₙ period ends and the T_{off} period starts (that is, when time T1 has elapsed since the transistor Tr was switched from OFF to ON) can be calculated by substituting t=T1 into Equation (6). This is because the current I has continuity as can be seen from FIG. 4. When replacing (1−e^{(-Z/L1×T1)}) with K (constant), the current I at t=T1 can be expressed as I(T1)=V¹_{IN}/Z×(1−e^{(-Z/L1×T1)})=K×V¹_{IN}/Z. Note that K satisfies the relationship of 0≤K<1, and when the value of Z/L1×T1 becomes sufficiently large, K can be approximated to 1.

Next, Equation (7): L1×dI/dt+Z×I=V¹_{IN}−V¹_{OUT} can be derived by Equations (1) and (3). Furthermore, by Equation (4), V²_{OUT} can be expressed by ∫Idt/C1+Vₛₜₐᵣₜ. Here, Vₛₜₐᵣₜ is the voltage of the capacitor C at the start of the T_{off} period (t=T1) and is a constant. If the threshold voltage of the diode D is V_{f}, Equation (8): V¹_{OUT}=V²_{OUT}+V_{f}=∫Idt/C1+Vₛₜₐᵣₜ+V_{f}=∫Idt/C1+V′ₒᵤₜ can be derived. Here, V′ₒᵤₜ=Vₛₜₐᵣₜ+V_{f} is a constant.

Furthermore, Equation (9): ∫Idt/C1+Z×I+L1×dI/dt=V¹_{IN}−V′ₒᵤₜ can be derived from Equations (7) and (8). By solving the differential equation of Equation (9), the current I during the T_{off} period can be expressed by a function of time t, the capacitor capacitance C1, the internal impedance Z, the inductance L1, V¹_{IN}, V′ₒᵤₜ, and K. When the start time of the T_{off} period is t=0, an initial value I(0) of the current I at that time is I(0)=K×V¹_{IN}/Z. When the T_{off} period ends (that is, time T2 elapses after the transistor Tr is switched from ON to OFF, and the current I becomes zero), I(T2)=0. The capacitor capacitance C1, the inductance L1, and V′ₒᵤₜ are constants, and when I(0) and T2 are measured, the values of V¹_{IN} and Z can be calculated.

Unlike the method described above, Z can be easily obtained. During the T_{off} period, since V¹_{OUT} is a voltage that is about 10 times larger than V²_{IN}, dI/dt also has a large value. In this case, ∫Idt in Equation (4) corresponds to the area of the triangle S in FIG. 4. Therefore, Equation (9): ∫Idt=K×V¹_{IN}/Z×T2/2=C1×ΔV is derived from Equation (4). Here, if the Tₒₙ time is sufficiently long, K≈1 can be approximated, and thus, when K=1 is substituted into Equation (9), Equation (10): V¹_{IN}/Z×T2/2=C1×ΔV is derived. C1 is a constant, and Z can be calculated from ΔV at the time point when the T_{off} period is sufficiently long (the time point when the current I becomes the minimum value), V¹_{IN} at the time point when the T_{off} period is sufficiently long (the time point when the current I becomes the minimum value), and T2 (the time when V¹_{OUT} becomes equal to V²_{IN}). Since V¹_{IN}=V²_{IN} at the time point when the T_{off} period is sufficiently long (the time point when the current I is consumed), V¹_{IN} can be specified by measuring V²_{IN}.

Note that the calculation of the internal impedance Z is executed by the control part.

(First Embodiment of Device)

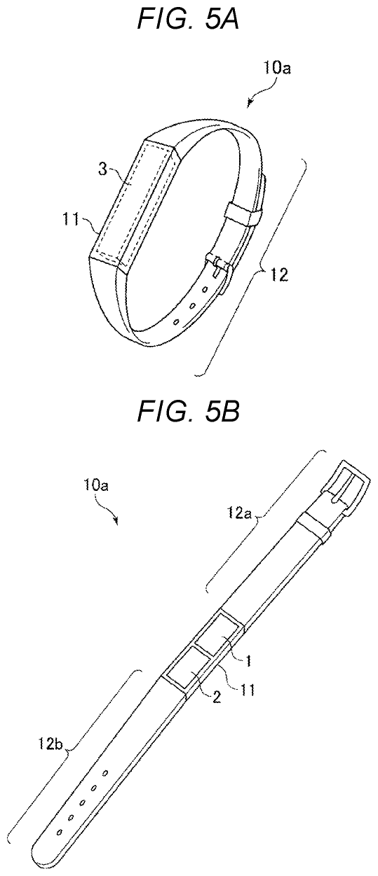

FIG. 5 is a diagram illustrating an example of a device according to an embodiment of the present invention. FIG. 5A is a diagram illustrating a shape when a device 10a is fixed to the body. Hereinafter, a surface that can be seen when the device 10a is fixed to the body is referred to as a front surface, and a surface that cannot be seen when the device 10a is fixed to the body is referred to as a back surface. FIG. 5B is a diagram illustrating a shape of the back surface of the device 10a.

As illustrated in FIG. 5, the device 10a has a shape like a wristwatch as a whole. The device 10a includes a main body part 11 including a functional part 3 and a fixing part 12 for fixing the device 10a to the body. In addition, as illustrated in FIG. 5B, a first conductive part 1 and a second conductive part 2 are provided on the back side of the main body part 11.

As described above, the first conductive part 1 and the functional part 3 are connected, the second conductive part 2 and the functional part 3 are connected, and the first conductive part 1 and the second conductive part 2 are not in contact with each other. Furthermore, as described above, the device 10a is energized by bringing the first conductive part 1 and the second conductive part 2 into contact with the body.

Although not illustrated, the main body part 11 may be provided with a hole, and the first conductive part 1 and the second conductive part 2 and the functional part 3 may be connected with a conductive wire or the like through the hole.

The shape of the main body part 11 is not limited to the example illustrated in FIG. 5, and may be any shape as long as it can include the functional part 3. For example, the shape of the main body part 11 may be a quadrangular flat shape as illustrated in FIG. 5, a polygonal flat shape, an elliptical flat shape, or a circular flat shape. Furthermore, the shape of the main body part 11 may be a three-dimensional shape instead of a flat shape, but the back surface of the main body part 11 provided with the first conductive part 1 and the second conductive part 2 is preferably substantially planar. Since the back surface of the main body part 11 has a flat shape, the first conductive part 1 and the second conductive part 2 easily come into contact with the body.

A material of the main body part 11 is not particularly limited, but is preferably a nonconductive material. For example, as a material of the main body part 11, a synthetic resin such as a phenol resin, a melamine resin, a urea resin, an alkyd resin, an epoxy resin, polyurethane, polyethylene, polypropylene, an acrylic resin, or polycarbonate can be used.

As described above, it is possible to provide a device including: a first conductive part and a second conductive part; and a functional part, wherein the first conductive part and the functional part are connected to each other, the second conductive part and the functional part are connected to each other, the first conductive part and the second conductive part are not in contact with each other, and the first conductive part and the second conductive part are brought into contact with a body to energize the device, so that the device can operate on a standalone power source.

As illustrated in FIG. 5B, the device 10a includes a fixing part 12a and a fixing part 12b on both sides of the main body part 11. Then, a mechanism similar to that of a general wristwatch, that is, coupling the fixing part 12a and the fixing part 12b via a component for coupling makes it possible to fix the wristwatch to the body. When the device 10a is fixed to the body, the device 10a has a shape as illustrated in FIG. 5A. In FIG. 5A, the fixing part 12a and the fixing part 12b are collectively referred to as a fixing part 12 in a state of being connected. By fixing the device 10a in this manner, the first conductive part 1 and the second conductive part 2 of the device 10a can be fixed in a state of being in contact with the body.

The shape of the fixing part 12 is not limited to the example illustrated in FIG. 5, and may be any shape as long as the device 10a can be fixed to the body. For example, the shape of the fixing part 12 may be of a ring shape like a bracelet. Alternatively, the shape of the fixing part 12 may be of a tape shape as described later.

The body part to which the device 10a is fixed is not particularly limited as long as the device 10a can be fixed by the fixing part 12. For example, the body part to which the device 10a is fixed may be a part that is thin to some extent, such as a wrist, an arm, an ankle, and a leg, and whose shape hardly changes even when the body is moved.

A material of the fixing part 12 is not particularly limited. As the material of the fixing part 12, a material similar to that of a general wristwatch can be used. For example, as the material of the fixing part 12, synthetic resin such as polyurethane, rubber, and silicon, synthetic fibers such as nylon, animal leather such as crocodile, calf, cordovan, lizard, pigskin, buffalo, galuchat, shark, ostrich, and python, synthetic leather such as polyester, metal such as stainless steel, titanium, and brass, and the like can be used. Alternatively, as the material of the fixing part 12, a material similar to that of a general medical tape as described later can be used.

As described above, the device includes the fixing part that fixes the first conductive part and the second conductive part in a state in which the first conductive part and the second conductive part are in contact with the body, whereby the device is easily fixed to the body and energized.

As illustrated in FIG. 5B, the device 10a includes the first conductive part 1 and the second conductive part 2 on the back side of the main body part 11. In FIG. 5B, the first conductive part 1 and the second conductive part 2 each having a rectangular sheet shape are provided on the back side of the main body part 11.

For the first conductive part 1 and the second conductive part 2, the above description can be adopted within a necessary range. For example, the first conductive part 1 and the second conductive part 2 may have flexibility.

Furthermore, when metal is used as the first conductive part 1 and the second conductive part 2, the first conductive part 1 may have a standard electrode potential different from that of the second conductive part 2. That is, different types of metals may be used for the first conductive part 1 and the second conductive part 2.

As described above, since the first conductive part 1 has a standard electrode potential different from that of the second conductive part 2, the direction in which the current flows can be made constant.

For the functional part 3 of the device 10a, the above description can be adopted within a necessary range.

The device 10a may include a voltage boost circuit as the functional part 3. Then, an electromotive force generated between the first conductive part 1 and the second conductive part 2 may be boosted by a voltage boost circuit.

As described above, the device includes the voltage boost circuit, and the voltage boost circuit boosts the electromotive force generated between the first conductive part and the second conductive part, so that a high voltage can be obtained even if the electromotive force is small.

The device 10a may include a measurement part that measures an internal impedance of the device 10a and/or a predetermined voltage in the device 10a, in the control part included in the functional part 3. For a method of measuring the internal impedance of the device and/or the predetermined voltage in the device, the foregoing description can be adopted within a necessary range.

The internal impedance of the device and/or the predetermined voltage in the device vary depending on the area where the first conductive part and the second conductive part of the device are in contact with the medium, the nature of the medium with which the first conductive part and the second conductive part of the device are in contact, and the like. For example, in a case where the amount of perspiration of a wearer of the device is small and a case where the amount of perspiration is large, the internal impedance of the device and/or the predetermined voltage in the device have different values. For example, in a case where the amount of electrolyte in the sweat of the wearer of the device is small and a case where the amount of electrolyte is large, the internal impedance of the device and/or the predetermined voltage in the device have different values.

As described above, the device includes the measurement part that measures the internal impedance of the device and/or the predetermined voltage in the device, so that the amount or the nature of the medium in contact with the first conductive part and the second conductive part of the device can be known. Then, the change in the state of the body of the wearer of the device can be known from the amount or the nature of the medium in contact with the first conductive part and the second conductive part of the device.

The device 10a may include a predetermined sensor as the functional part 3. Then, the sensor may be operated by energization by bringing the first conductive part 1 and the second conductive part 2 of the device 10a into contact with the body.

The type of the sensor is not particularly limited as long as the sensor senses or measures predetermined information. For example, the type of sensor may be one that senses or measures a heart rate, cardiac potential, blood pressure, body temperature, or the like of a wearer of the device. Alternatively, the type of the sensor may be one that senses or measures acceleration, outside temperature, atmospheric pressure, illuminance, ultraviolet irradiation amount, and the like.

As described above, the device includes the predetermined sensor, and the sensor operates by energization by bringing the first conductive part and the second conductive part into contact with the body, so that the state of the body of the wearer of the device and/or the state of the environment outside the wearer of the device can be known.

Furthermore, the device 10a may include, as the functional part 3, a communication part that transmits an internal impedance of the device 10a and/or a predetermined voltage in the device 10a measured by the measurement part, or information acquired by a predetermined sensor (hereinafter, referred to as device acquisition information) to another computer device. Furthermore, the device 10a may have a clock function as the functional part 3. Then, the device 10a may transmit the information regarding the time to another computer device together with the device acquisition information.

The computer device is not particularly limited as long as it includes a communication part and a control part, and it is, for examples, a server device, a terminal device, or the like. When the computer device is a terminal device, it is preferable that a dedicated application corresponding to the device of the present invention is installed.

In addition, the computer device may include a storage part. Then, the storage part preferably stores the device acquisition information and the information regarding the time received by the communication part.

Furthermore, the computer device may include an input part. Then, information regarding the physical condition of the wearer of the device may be able to be input to the computer device. Since the information regarding the physical condition of the wearer of the device can be input to the computer device, the information on the relevance between the physical condition of the wearer of the device and the device acquisition information can be obtained. In addition, the storage part of the computer device may store the device acquisition information of the wearer of the device at normal times and the device acquisition information of the wearer of the device at the time of poor physical condition in the past.

Furthermore, the computer device may include a display part or a sound processing part. Then, when the device acquisition information received by the computer device is different from the device acquisition information at normal times, or when the device acquisition information received by the computer device is similar to the device acquisition information at the time of poor physical condition in the past, the information may be displayed on the display part of the computer device or the notification sound may be transmitted. Furthermore, the details of the device acquisition information and the time information may be displayed on the display part of the computer device by the user's operation.

As described above, the device includes the communication part that transmits to another computer device the internal impedance of the device and/or the predetermined voltage in the device measured by the measurement part or the information acquired by the predetermined sensor, so that the change in the state of the body of the wearer of the device, the state of the body of the wearer of the device, and/or the state of the external environment of the wearer of the device can be confirmed in the other computer device.

Alternatively, the device 10a may include a measurement part and a predetermined sensor, and the device acquisition information may include both the internal impedance of the device 10a and/or the predetermined voltage in the device 10a measured by the measurement part and the information acquired by the predetermined sensor.

Furthermore, the device 10a may have various functions other than the above. For example, the device 10a may include a display part, and the time and the device acquisition information may be displayed on the display part. Alternatively, the device 10a may include an electrical stimulation generator, an electrical stimulation connector, and an electrical stimulation provider as described later.

In addition, the device 10a preferably has a waterproof function.

(Second Embodiment of Device)

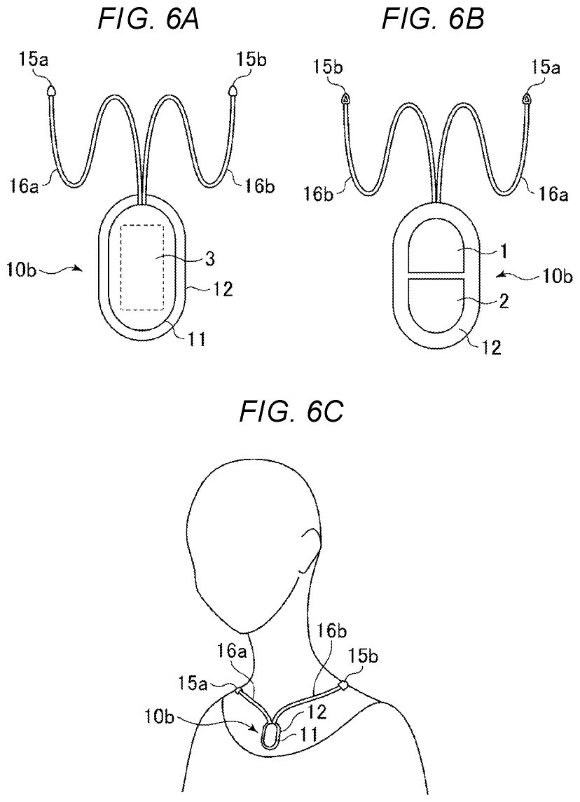

FIG. 6 is a diagram illustrating examples of a device according to an embodiment of the present invention. FIG. 6A is a diagram illustrating a shape of a surface of a device 10b. FIG. 6B is a diagram illustrating a shape of a back surface of the device 10b. FIG. 6C is a diagram illustrating an attachment example of the device 10b.

As illustrated in FIG. 6, the device 10b includes a main body part 11 including a functional part 3, a fixing part 12 that fixes the device 10b to a body, an electrical stimulation provider 15 that provides an electrical stimulation to the body, and an electrical stimulation connector 16 that connects an electrical stimulation generator to be described later and the electrical stimulation provider 15. In addition, as illustrated in FIG. 6B, the fixing part 12 includes a first conductive part 1 and a second conductive part 2.

As described above, the first conductive part 1 and the functional part 3 are connected, the second conductive part 2 and the functional part 3 are connected, and the first conductive part 1 and the second conductive part 2 are not in contact with each other. Furthermore, as described above, the device 10b is energized by bringing the first conductive part 1 and the second conductive part 2 into contact with the body.

Although not illustrated, two conductive parts connected to the functional part 3 may be provided on the back surface of the main body part 11, and the first conductive part 1 and the second conductive part 2 may be connected to the functional part 3 by bringing the first conductive part 1 and the second conductive part 2 into contact with each of the conductive parts.

The shape of the main body part 11 is not limited to the example illustrated in FIG. 6, and may be any shape as long as it can include the functional part 3. For example, the shape of the main body part 11 may be an elliptical flat shape as illustrated in FIG. 6, a circular flat shape, a quadrangular flat shape, or a polygonal flat shape. Furthermore, the shape of the main body part 11 may be a three-dimensional shape instead of a flat shape, but the back surface of the main body part 11 is preferably substantially planar. Since the back surface of the main body part 11 is substantially planar, the device 10b can be easily worn on the body.

For the material of the main body part 11, the description of the device 10a can be adopted within a necessary range.

As described above, it is possible to provide a device including: a first conductive part and a second conductive part; and a functional part, wherein the first conductive part and the functional part are connected to each other, the second conductive part and the functional part are connected to each other, the first conductive part and the second conductive part are not in contact with each other, and the first conductive part and the second conductive part are brought into contact with a body to energize the device, so that the device can operate on a standalone power source.

As illustrated in FIG. 6B, the device 10b includes a fixing part 12 on the back surface of the main body part 11. The fixing part 12 of the device 10b has a tape shape, and as illustrated in FIG. 6C, the device 10b can be fixed to the body. The fixing part 12 of the device 10b preferably has adhesive surfaces on both sides. In addition, among the adhesive surfaces of the fixing part 12, a surface to be attached to the main body part 11, that is, the front surface preferably has a strong adhesive force, and a surface to be attached to the body, that is, the back surface preferably has a weak adhesive force. The first conductive part 1 and the second conductive part 2 are provided on a surface to be attached to the body out of the adhesive surfaces of the fixing part 12. By fixing the device 10b as illustrated in FIG. 6C, the first conductive part 1 and the second conductive part 2 of the device 10b can be fixed in a state of being in contact with the body.

The fixing part 12 of the device 10b is not particularly limited as long as it has flexibility and adhesiveness. For example, a medical tape or the like can be used as the fixing part 12 of the device 10b. It is preferable that the fixing part 12 of the device 10b is less likely to cause rash.

The shape of the fixing part 12 is not limited to the example illustrated in FIG. 6, and may be any shape as long as the device 10b can be fixed to the body. For example, the shape of the fixing part 12 may be an elliptical shape as illustrated in FIG. 6, a circular shape, a quadrangular shape, or a polygonal shape.

The body part to which the device 10b is fixed is not particularly limited as long as the device 10b can be fixed by the fixing part 12. For example, the body part to which the device 10b is fixed may be a three-dimensional part, such as a neck, a chest, an abdomen, a back, and a waist, whose shape is easily changed by moving the body. Since the fixing part 12 of the device 10b has a tape shape and has flexibility and adhesiveness, the device 10b can be fixed to the body even at such a part.

The material of the fixing part 12 is not particularly limited as long as the device 10b can be fixed to the body. For example, as the material of the fixing part 12, a material similar to that of a general medical tape can be used. Specifically, polyester, nonwoven fabric, or the like can be used as the material of the support of the fixing part 12. In addition, specifically, synthetic rubber, acryl, or the like can be used as a material of the adhesive surface of the fixing part 12.

As described above, the device includes the fixing part that fixes the first conductive part and the second conductive part in a state in which the first conductive part and the second conductive part are in contact with the body, whereby the device is easily fixed to the body and energized.

As illustrated in FIG. 6B, the fixing part 12 of the device 10b includes the first conductive part 1 and the second conductive part 2. In FIG. 6B, the fixing part 12 of the device 10b is provided with the first conductive part 1 and the second conductive part 2 each having a semi-elliptical sheet shape.

The first conductive part 1 and the second conductive part 2 may be integrally formed with the fixing part 12. That is, the fixing part 12 may be provided around the first conductive part 1 and the second conductive part 2, and the entire shapes of the first conductive part 1 and the second conductive part 2 may be visible from both the back surface and the front surface of the fixing part 12. Then, when the fixing part 12 is provided on the back surface of the main body part 11 of the device 10b, the first conductive part 1 and the second conductive part 2 may be connected to the functional part 3.

Alternatively, the first conductive part 1 and the second conductive part 2 may be formed separately instead of being integrated with the fixing part 12. That is, the first conductive part 1 and the second conductive part 2 may be provided on the adhesive surface on the back surface of the fixing part 12, and the entire shapes of the first conductive part 1 and the second conductive part 2 can be visible from the back surface of the fixing part 12, but the entire shapes of the first conductive part 1 and the second conductive part 2 may not be visible from the front surface of the fixing part 12. In this case, when the fixing part 12 is provided on the back surface of the main body part 11 of the device 10b, a hole is preferably provided in the fixing part 12 so that the first conductive part 1 and the second conductive part 2 are connected to the functional part 3.

The first conductive part 1 and the second conductive part 2 of the device 10b may have flexibility. In a case where the fixing part 12, and the first conductive part 1 and the second conductive part 2 both have flexibility, the first conductive part 1 and the second conductive part 2 can be fixed in a state of being in contact with the body even in a three-dimensional part of the body such as the neck, the chest, the abdomen, the back, and the waist.

As described above, since the first conductive part and the second conductive part of the device have flexibility, the first conductive part and the second conductive part can come into contact with the body at various places of the body. The device can then be energized at various locations on the body.

Furthermore, when metal is used as the first conductive part 1 and the second conductive part 2, the first conductive part 1 may have a standard electrode potential different from that of the second conductive part 2. That is, different types of metals may be used for the first conductive part 1 and the second conductive part 2.

As described above, since the first conductive part 1 has a standard electrode potential different from that of the second conductive part 2, the direction in which the current flows can be made constant.

In addition, for the first conductive part 1 and the second conductive part 2, the above description can be adopted within a necessary range.

The fixing part 12 and/or the first conductive part 1 and the second conductive part 2 of the device 10b may be discarded after use and may be replaced with a new one. In this case, the fixing part 12 and/or the first conductive part 1 and the second conductive part 2 may be discarded every time they are used, or may be discarded after they are used a plurality of times.

For the functional part 3 of the device 10b, the above description can be adopted within a necessary range.

The device 10b may include a voltage boost circuit as the functional part 3. Then, an electromotive force generated between the first conductive part 1 and the second conductive part 2 may be boosted by a voltage boost circuit.

As described above, the device includes the voltage boost circuit, and the voltage boost circuit boosts the electromotive force generated between the first conductive part and the second conductive part, so that a high voltage can be obtained even if the electromotive force is small.

The device 10b may include a measurement part that measures an internal impedance of the device 10b and/or a predetermined voltage in the device 10b, in the control part included in the functional part 3. For a method of measuring the internal impedance of the device and/or the predetermined voltage in the device, the foregoing description can be adopted within a necessary range.

The internal impedance of the device and/or the predetermined voltage in the device vary depending on the area where the first conductive part and the second conductive part of the device are in contact with the medium, the nature of the medium with which the first conductive part and the second conductive part of the device are in contact, and the like. For example, in a case where the amount of perspiration of a wearer of the device is small and a case where the amount of perspiration is large, the internal impedance of the device and/or the predetermined voltage in the device have different values. For example, in a case where the amount of electrolyte in the sweat of the wearer of the device is small and a case where the amount of electrolyte is large, the internal impedance of the device and/or the predetermined voltage in the device have different values.

As described above, the device includes the measurement part that measures the internal impedance of the device and/or the predetermined voltage in the device, so that the amount or the nature of the medium in contact with the first conductive part and the second conductive part of the device can be known. Then, the change in the state of the body of the wearer of the device can be known from the amount or the nature of the medium in contact with the first conductive part and the second conductive part of the device.

The device 10b may include a predetermined sensor as the functional part 3. Then, the sensor may be operated by energization by bringing the first conductive part 1 and the second conductive part 2 of the device 10b into contact with the body.

For the type of the sensor, the description of the device 10a can be adopted within a necessary range.

As described above, the device includes the predetermined sensor, and the sensor operates by energization by bringing the first conductive part and the second conductive part into contact with the body, so that the state of the body of the wearer of the device and/or the state of the environment outside the wearer of the device can be known.

Furthermore, the device 10b may include, as the functional part 3, a communication part that transmits the device acquisition information to another computer device. Furthermore, the device 10b may have a clock function as the functional part 3. Then, the device 10b may transmit the information regarding the time to another computer device together with the device acquisition information.

For the computer device, the description of the device 10a can be adopted within a necessary range.

As described above, the device includes the communication part that transmits to another computer device the internal impedance of the device and/or the predetermined voltage in the device measured by the measurement part or the information acquired by the predetermined sensor, so that the change in the state of the body of the wearer of the device, the state of the body of the wearer of the device, and/or the state of the external environment of the wearer of the device can be confirmed in the other computer device.

Alternatively, the device 10b may include a measurement part and a predetermined sensor, and the device acquisition information may include both the internal impedance of the device 10b and/or the predetermined voltage in the device 10b measured by the measurement part and the information acquired by the predetermined sensor.

In addition, the device 10b may include, as the functional part 3, an electrical stimulation generator that generates a current for giving an electrical stimulation to the body by a voltage generated by bringing the first conductive part 1 and the second conductive part 2 into contact with the body. In addition, as illustrated in FIG. 6, the device 10b may include an electrical stimulation provider 15 that provides an electrical stimulation to the body, and an electrical stimulation connector 16 that connects the electrical stimulation provider 15 and the electrical stimulation generator.

In FIG. 6, the device 10b includes two electrical stimulation providers 15 and two electrical stimulation connectors 16. The electrical stimulation generator and an electrical stimulation provider 15a are connected by an electrical stimulation connector 16a, and the electrical stimulation generator and an electrical stimulation provider 15b are connected by an electrical stimulation connector 16b.

The device 10b may include one or more electrical stimulation providers 15 and one or more electrical stimulation connectors 16. For example, two, three, five, or more may be included.

In the device 10b, for example, as illustrated in FIG. 6C, the main body part 11 can be worn on the chest of the body using the fixing part 12, and the electrical stimulation providers 15a and 15b can be worn on both shoulders of the body one by one.

Mounting the device 10b in this way causes the first conductive part 1 and the second conductive part 2 provided in the fixing part 12 to come into contact with the body, which generates a voltage. The generated voltage causes the electrical stimulation generator to generate a current for giving an electrical stimulation to the body. The current generated by the electrical stimulation generator is transmitted to the electrical stimulation provider 15 by the electrical stimulation connector 16. Then, the electrical stimulation provider 15 provides the body with the current generated by the electrical stimulation generator.

The electrical stimulation generator is not particularly limited as long as it generates the current for giving an electrical stimulation to the body. For example, as the electrical stimulation generator, an electrical stimulation generator used in an electric therapy device or the like can be used.

The device 10b may include an input part, and the magnitude of the current generated by the electrical stimulation generator may be adjusted by the input part. The input part may switch ON/OFF of the current generated by the electrical stimulation generator.

The electrical stimulation connector 16 is not particularly limited as long as it electrically connects the electrical stimulation generator and the electrical stimulation provider 15. For example, a cord in which a conductive wire is covered with an insulator can be used as the electrical stimulation connector 16.

The electrical stimulation provider 15 is not particularly limited as long as it provides the body with the current generated by the electrical stimulation generator to provide the body with an electrical stimulation. The electrical stimulation provider 15 may have conductivity. The electrical stimulation provider 15 may have flexibility and adhesiveness. Alternatively, the electrical stimulation provider 15 may be a combination of a portion having conductivity and a portion having flexibility and adhesiveness.

Examples of the material of the electrical stimulation provider 15 include metal, conductive polymer, carbon, conductive fibers, and conductive rubber.

Furthermore, for the electrical stimulation provider 15, a nonconductive material covered with a conductive material, a nonconductive material mixed with a conductive material, or the like may be used. For example, a plastic film coated with a metal or cream-like paste or a gel-like material mixed with metal powder may be used.

The shape of the electrical stimulation provider 15 is not particularly limited. The electrical stimulation provider 15 may have a rectangular parallelepiped shape, a cylindrical shape (rod shape), a pyramid shape, a conical shape, a plate shape, a sheet shape, a film shape, a needle shape, a string shape, or powder form, and may have any shape.

Furthermore, the electrical stimulation provider 15 may be shaped like a minute needle for acupuncture. In addition, the electrical stimulation provider 15 may be a minute needle for acupuncture fixed to a medical tape.

Alternatively, as illustrated in FIG. 6C, the electrical stimulation provider 15 may not provide an electrical stimulation to the body percutaneously, but may be implanted under the skin to provide electrical stimulation to organs and nerves in the body. In this case, the device 10b may also be implanted under the skin.

As described above, since the device includes the electrical stimulation generator that generates a current for giving an electrical stimulation to the body by the voltage generated by bringing the first conductive part and the second conductive part into contact with the body, the electrical stimulation can be given to the body by the voltage obtained from the body.

Furthermore, the device 10b may have various functions other than the above. For example, the device 10b may include a display part, and time and device acquisition information may be displayed on the display part.

In addition, the device 10b preferably has a waterproof function.

In the embodiments of the present invention, the “conductive part” may be, for example, made of any material as long as it is an energizable member. The “functional part” is, for example, a part that executes a predetermined function by energization. The function may be to convert electricity into energy such as light or heat, or to control a circuit.

In the embodiments of the present invention, the “electrolytic solution” refers to, for example, a solution having electrical conductivity in which an ionic substance is dissolved in a polar solvent. The “voltage boost circuit” refers to, for example, a circuit that boosts and outputs an input voltage. The “voltage step-down circuit” refers to, for example, a circuit that steps down and outputs an input voltage. The “conductive polymer” refers to, for example, a polymer compound having electrical conductivity. “Carbon” refers to, for example, carbon fiber having conductivity. The “integrally forming” refers to, for example, joining different objects to each other, and more specifically, joining by a chemical and/or physical force such as adhesion using an adhesive, mechanical joining using another member, welding, and pressure bonding can be mentioned.

### REFERENCE EXAMPLES

Hereinafter, the present invention will be described in more detail with reference to reference examples, but the present invention is not limited by these reference examples at all.

### Reference Example 1

The following test was performed at normal temperature and normal pressure. A system was constructed using a device including configurations of the first conductive part 1, the second conductive part 2, and the functional part 3 illustrated in FIG. 1 and a medium. A plate-shaped member (a thickness of 0.5 mm, 10 cm×15 cm) made of stainless steel (austenite, SUS 304 series) was used as the first conductive part 1, a plate-shaped member (a thickness of 0.5 mm, 10 cm×15 cm) made of a galvanized steel plate (iron) was used as the second conductive part 2, and the first conductive part 1, the second conductive part 2, and the functional part 3 were connected to each other by copper conductive wires. The functional part 3 includes an electric consumption part, an output voltage conversion part, and a control part. In addition, the functional part 3 having an input impedance of 1 kΩ or more and a non-linear current-voltage characteristic was used. As the electric consumption part, an LED bulb that lights up when a current of 2 mA or more flows was used. The voltage boost circuit illustrated in FIG. 2A was used as the output voltage conversion part to form a system.

The first conductive part 1 was connected to the input terminal A1 of the voltage boost circuit of the output voltage conversion part, and the output terminal B1 of the voltage boost circuit was connected to the LED bulb. Furthermore, the second conductive part 2 was connected to the input terminal A2 of the voltage boost circuit, and the output terminal B2 of the voltage boost circuit was connected to a terminal opposite to the terminal connected to the output terminal B1 of the LED bulb.

Pure water (KOGA Chemical Mfg Co., Ltd., high-purity purified water, temperature 25° C.: medium) was placed in an acrylic container (cubic body with outer diameter of 15 cm×15 cm×15 cm, inner diameter of 14.5 cm) up to a height of 7.5 cm, and the first conductive part 1 and the second conductive part 2 were immersed in the pure water to construct the system. The first conductive part 1 and the second conductive part 2 were not in contact with each other, the distance between the first conductive part 1 and the second conductive part 2 was 12 cm, and the first conductive part 1 and the second conductive part 2 were installed so that plate-like planes of the first conductive part 1 and the second conductive part 2 were parallel to each other.

For the constructed system, the voltage between the first conductive part 1 and the second conductive part 2 was measured (Measurement 1). For the measurement, a 34401A multimeter manufactured by Agilent Technologies was used. The result is shown in Table 1. In the system shown in Reference Example 1, the LED bulb repeatedly blinked every 270 to 330 seconds. That is, it was confirmed that electromotive force was generated from the first conductive part 1 and/or the second conductive part 2.

Next, the first conductive part 1 and the second conductive part 2 were immersed, and pure water (KOGA Chemical Mfg Co., Ltd., high-purity purified water, temperature 25° C.: medium) was placed in an acrylic container (cubic body with outer diameter of 15 cm×15 cm×15 cm, inner diameter of 14.5 cm) up to a height of 7.5 cm, and the first conductive part 1 and the second conductive part 2 were immersed in the pure water. The first conductive part 1 and the second conductive part 2 were not in contact with each other, the distance between the first conductive part 1 and the second conductive part 2 was 12 cm, and the first conductive part 1 and the second conductive part 2 were installed so that plate-like planes of the first conductive part 1 and the second conductive part 2 were parallel to each other. The first conductive part 1 and the second conductive part 2 were not electrically connected. Then, the voltage between the first conductive part 1 and the second conductive part 2 was measured using a 34401A multimeter (Measurement 2). Furthermore, in this state, the resistance value of the medium between the first conductive part 1 and the second conductive part 2 was measured (Measurement 3).

### Reference Example 2

Measurements 1 to 3 were performed in the same manner as in Example 1 except that the medium was changed to soil (soil of house plant manufactured by PROTOLEAF, Inc.). The results are shown in Table 1. In the system shown in Reference Example 2, the LED bulb repeatedly blinked at substantially equal intervals every 21 to 23 seconds. That is, it was confirmed that electromotive force was generated from the first conductive part 1 and/or the second conductive part 2.

### Reference Example 3

Measurements 1 to 3 were performed in the same manner as in Reference Example 1 except that a waste cloth soaked in an aqueous solution in which 5 g of salt (Coarse Salt produced by Hakata Salt Co., Ltd.) was dissolved in 50 g of pure water (the same as in Reference Example 1) was attached to the surfaces of the first conductive part 1 and the second conductive part 2 in contact with the medium, and the medium was changed to sand (silica sand having a particle size peak (weight ratio) of about 0.9 mm manufactured by Toyo Matelan Corporation). The results are shown in Table 1. In the system shown in Reference Example 3, the LED bulb repeatedly blinked every 80 to 100 seconds. That is, it was confirmed that electromotive force was generated from the first conductive part 1 and/or the second conductive part 2.

TABLE 1

| | Measurement 1 | Measurement 2 | Measurement 3 |
|---|---|---|---|
| | [mV] | [mV] | [kΩ] |
| Reference Example 1 | 239 | 952 | 20 |
| Reference Example 2 | 291 | 822 | 1,700 |
| Reference Example 3 | 253 | 954 | 250 |

### Reference Example 4

Pure water placed in an acrylic container up to a height of 7.5 cm in Reference Example 1 was added up to a height of 10 cm. By adding pure water, it was possible to confirm a change in the internal impedance of the system described above. In addition, by adding pure water, a change in the input voltage V²_{IN} when the T_{off} period started was able to be confirmed. The internal impedance was calculated by the above-described calculation method.

### Reference Example 5

Pure water placed in an acrylic container up to a height of 7.5 cm in Reference Example 1 was added up to a height of 10 cm over 5 minutes. It was possible to confirm that the change amount per unit time of the internal impedance of the system described above changed. In addition, it was possible to confirm that the change amount per unit time of the input voltage was changed by adding pure water. The internal impedance was calculated by the above-described calculation method. The input voltage is the input voltage V²_{IN} when the T_{off} period starts.

### REFERENCE SIGNS LIST

1 First conductive part
2 Second conductive part
3 Functional part
10 Device
11 Main body part
12 Fixing part
15 Electrical stimulation provider
16 Electrical stimulation connector

1. A device comprising:
a first conductive part and a second conductive part; and
a functional part, wherein
the first conductive part and the functional part are connected to each other,
the second conductive part and the functional part are connected to each other,
the first conductive part and the second conductive part are not in contact with each other; and
the device is energized by bringing the first conductive part and the second conductive part into contact with a body.

2. The device according to claim 1, comprising
a voltage boost circuit, wherein
an electromotive force generated between the first conductive part and the second conductive part is boosted by the voltage boost circuit.

3. The device according to claim 1, wherein the first conductive part and the second conductive part have flexibility.

4. The device according to claim 1, comprising
a measurement part that measures an internal impedance of the device and/or a predetermined voltage in the device.

5. The device according to claim 1, comprising
a predetermined sensor, wherein
the sensor is operated by bringing the first conductive part and the second conductive part into contact with the body to be energized.

6. The device according to claim 4, comprising
a communication part that transmits to another computer device the internal impedance of the device and/or the predetermined voltage in the device measured by the measurement part.

7. The device according to claim 1, wherein the first conductive part has a standard electrode potential different from that of the second conductive part.

8. The device according to claim 1, comprising
a fixing part for fixing the first conductive part and the second conductive part in a state of being in contact with the body.

9. The device according to claim 1, comprising
an electrical stimulation generator that generates a current for giving an electrical stimulation to the body by a voltage generated by bringing the first conductive part and the second conductive part into contact with the body.

10. An energizing method comprising energizing a device,
the device including a first conductive part and a second conductive part, and a functional part, the first conductive part and the functional part being connected to each other, the second conductive part and the functional part being connected to each other, the first conductive part and the second conductive part being not in contact with each other,
by bringing the first conductive part and the second conductive part into contact with a body.

11. The device according to claim 5, comprising
a communication part that transmits to another computer device information acquired by the predetermined sensor.
